(19) United States
(12) Patent Application Publication
(10) Pub. No.: US 2010/0151474 A1
(43) Pub. Date: Jun. 17, 2010

(54) Multifunctional Device For Diagnostics and Method For Testing Biological Objects

(76) Inventors: Vladimir Nikolaevich Afanasyev, Puschino (RU); Gaida Vladislavovna Afanasyeva, Puschino (RU); Sergey Vladimirovich Biryukov, Puschino (RU); Igor Petrovich Beletsky, Puschino (RU)

Correspondence Address: John Alumit, 16830 Ventura Blvd. Suite 360, Encino, CA, 91436, US

(21) Appl. No.: 12/645,472

(22) Filed: Dec. 22, 2009

(30) Foreign Application Priority Data
Jun. 25, 2007 (RU) 2007123475
Jun. 25, 2007 (RU) 2007123476
Jun. 25, 2007 (RU) 2007123477

(51) Int. Cl. C12Q 1/68; C12M 1/34; C12Q 1/02

(52) U.S. Cl. 435/6; 435/288.7; 435/287.9; 435/287.2; 435/29

(57) ABSTRACT

A multifunctional device for measuring fluorescence, luminescence and light transmission for diagnostics. A sample carrier is designed in the form of a biochip, cell, pan or microplate. The device comprises a first and second group of screens mounted behind the rear surface of a sample solid carrier. Light sources of the sample are provided with light-absorbing elements for suppressing light reflected from the front surface of the sample carrier and from screen surfaces. Screen holders allow for alternatively mounting light reflective/retroreflective screens to maximize fluorescent or luminescent signal. A diffusing screen measures light transmission through the sample. Light-absorbing screens behind the rear surface of the sample and light-absorbing elements on light sources from the sample's top surface, increase signal-to-noise ratio. Said device permits measuring signals on biochip surfaces and in solutions during hybridization or amplification reactions. The device and diagnostic method are suitable for mass screening of biological material samples.

### CROSS-REFERENCE TO RELATED APPLICATION

This application claims the benefit of the priority filing date of international application no. PCT/RU2008/000389, publication no. WO/2009/002225 and Russian application nos. 2007123475 filed on Jun. 25, 2007, 2007123476 filed on Jun. 25, 2007, and 2007123477 filed on Jun. 25, 2007.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

This invention discloses a device for scanning diagnostics results in the fields of medicine, veterinary, food products control, crime detection and other diagnostics fields related to identifying biologically active agents. More specifically the invention pertains to scanning devices for different types of objects layered onto a solid carrier, for example, as biochips or recording devices for biological objects in solutions placed in cells, multiboards or hybridization chambers, chromatographic carriers, and gels.

Many engineering solutions are available in the formation and recording of signals obtained during diagnostics of biological specimens. Colorimetric or fluorescent markers are most frequently used to record the signal and identify the objects.

Of wide use are highly specialized optical devices designed to operate in a mode of measuring the optical signal interacting with the specimen for analysis. Such modes may include optical transmission measuring, measuring the reflectance signal from the specimen surface, measuring fluorescence and luminescence levels, or measuring the signal of resonance interaction of molecules at BRET [1] or FRET modes.

In diagnostics devices biological specimens may be layered onto a solid carrier, for example, designed as slides [2] or biochips [3]. In certain embodiments, biological specimens are studied in solutions placed in open cells of multiboards [1] or in hermetic cells, for example, for performing hybridization [4].

In general multifunctional devices are used for mass screening in large testing laboratories, clinics or research laboratories.

Multifunctional devices for measuring luminescence and fluorescence levels as well as absorption are known [5]. The tested specimen can be placed in a cell or microboard. The device contains a monochromator and multiple optical filters. Transmission of optical signals via optical fibers allows for converting optical systems for operation at different modes. The device has a mechanism for relocating microboard position, monitored by a processor that also controls the wavelength.

Known is the method [6] according to which the specimen is detected at different modes and the detection result is estimated for one or multiple specimens. The results of the analysis are obtained using photoluminescence, chemiluminescence, absorption, or light scattering measurements. The device is designed as a complex of blocks. Light guides are used to transmit optical signals. The device is designed to perform measurements in microboards with multiple individual cells.

In the device [7], optical fibers and optical elements form a system of illumination of a specimen placed in microboards as well as a system of optical signal measurements upon measuring fluorescence and luminescence levels and light absorption. To make measurements in various cells of microboards, computer-monitored double-coordinate displacement is used.

The device for measuring luminescence and fluorescence radiation [1] is known, which allows developing at least three modes of luminescence and fluorescence excitation, and signal absorption, due to the formation of different optical systems. A general disadvantage of the above described devices may include a great number of optical elements, complexity of the design, the requirement for using precision mechanisms to replace objects in the X-Y coordinates, mechanisms for switching the filters and the optical signal path, as well as the loss of optical signals upon signal transmission via optical fiber.

Another group of devices for diagnostics of biological objects is connected with plotting designs which permit measurements of fluorescent emission signals in a real-time mode, for example, in hybridization or amplification analysis.

Known is the optical instrument [8] for monitoring PCR in cells placed in a temperature-controlled unit. The emitted light flux from every cell formed using a Fresnel lens is recorded by a charge-coupled detector (CCD). The device for amplification and detection of nucleic acids in real-time mode is known [9, 10]. The device for in-situ detection of luminescent radiation of biological objects is known [2]. In this device, a driving gear is used that relocates the specimen in the X-Y coordinates monitored by computer. The device for nucleic acid hybridization on the solid surface of biochips placed within a liquid cell is known [4]. The optical system of illumination of the biochip working surface is based on the dark field principle.

The described systems of measuring optical signals in real-time mode pertain to highly specialized devices, and are not intended for work with biological specimens immobilized on biochips. They do not contain elements contributing to increasing the signal-to-noise ratio and are designed using typical schemes.

A great number of scanners or microscopes are known operating as confocal ones in which an ultraviolet radiation flux is formed incident upon the front side of the surface of the object solid carrier and causing fluorescence of the specimen [3, 11-18]. To form a confocal image, the instrument contains many additional elements, gives small-sized images and requires relocation in the X-Y coordinates of the solid carrier, on which the object for analysis is layered, or relocation of the optical system relative to the immobilized object.

Devices are known in which fluorescent radiation is formed by transmitting the ultraviolet light flux from the back side of the biochip via a transparent solid carrier to the receiving CCD array. The UV-light flux is incident upon the carrier surface perpendicular to the biochip surface [19] or at an angle to the carrier surface [20].

Optical scanners and microscopes are known in which the dark field principle of image formation is used to control the surface [21-22], including fluorescence recording [23-26]. However, in the devices described above, little attention is paid to increasing the signal-to-noise ratio and to the possibility of working at several measuring modes, e.g., the possibility of measuring light flux fluorescence and absorption upon recording colorimetric markers. In many optical systems, a narrow light beam is formed which requires using devices for slide or biochip relocation in the X-Y coordinates.

To decrease the level of stray light and increase the signal-to-noise ratio, absorbing elements are introduced to optical systems. It is known from the background of the invention that absorbing elements are used in optical systems for quality control of the surface of semi-conductor plates [27-28], in optical systems of confocal microscopes [29], for cell control in flow systems employing measurements of the reflected fluorescent signal [30].

In some devices, the problem of increasing the signal level is also solved by the formation of double transmission of the light flux via the studied specimen. Microscopes are known [31-32] in which the light beam is transmitted twice via the studied specimen with the use of two objective lenses having identical optical characteristics and an additional mirror placed on the back side of the object for analysis to reflect the light flux passing via the object for analysis. The confocal laser microscope is known [33] in which an angle reflector is placed on the back side of the object for analysis and lenses are placed on the front and rear surfaces of the object for analysis in order to form a parallel-sided light beam incident upon the angle reflector that returns the incident light and directs it to the object for analysis, thus increasing the image contrast.

The engineering solution closest to the described invention is given in RU patent 2182328 [24]. A microscope allows for working in dark field mode when measuring fluorescence signals and in the mode of a light flux passing via a transparent carrier of the specimen. Disadvantages of this system are a small working illumination field (the diameter of about 10 mm) and the influence of scattered radiation deteriorating the signal-to-noise ratio.

The analysis of the background of the invention has demonstrated that there is a need to develop a simpler and more reliable device for working in different modes of diagnostics, including real-time modes with improved characteristics of the signal-to-noise ratio.

A task of the invention is to maximize simplification and to reduce the price of its optical system while retaining the possibility to transform optical systems for choosing different modes of operation of the device upon scanning of the studied specimens, and with a simultaneous increase of the signal-to-noise ratio.

Another task is an increase of scanning efficiency by designing an optical system allowing the formation of illumination of a maximally large working field of a biochip, cell or microboard.

The next task of this invention is the development of a device design that provides an opportunity to work not only in a multi-mode process, but also an opportunity to measure parameters of specimens placed in different media or immobilized on different surfaces in real-time mode.

The object of this invention is a device for scanning the diagnostics results and a method for performing the diagnostics.

In the described invention, the possibility of changing the diagnostics modes is realized by mounting or changing the elements located along the axis of the optical system, and/or along light beams formed by light sources. To that end, the mounted elements are made as screens with an absorbing, reflecting, retroreflective or light-scattering surface, which permits improving the signal-to-noise ratio by decreasing the stray light background and/or increasing the level of the signal measured. An additional increase of the signal-to-noise ratio is caused by the light-absorbing surface placed on the surface of the specimen illuminators.

The device contains sources of optical radiation forming the illumination of the working field, an optical system, a detector, an attachment point of the specimen holder, and a solid carrier of the specimen studied. To solve the posed tasks, the device has at least two light sources forming the illumination of the working field, an optical system, a detector, an attachment point of the specimen holder, a solid carrier of the specimen studied. In addition, the device has a first and second groups of screens, the first group containing at least one screen, and the second group containing at least two screens. The screens of the first and second groups are mounted at the rear surface of the solid carrier of the specimen, and the illuminators mounted above the working surface of the specimen carrier are equipped with absorbing elements for suppressing the reflected light from the front surface of the carrier and the screen surfaces. The screens of the first group are positioned perpendicular to the axis of the recording system, while the screens of the second group are positioned perpendicular to the optical axes of the illuminators.

Another feature of this invention is that the front surface of the first screen from the first group is designed so that it can reflect or return the light fluxes emitted from the first and second illuminators. The holder of a specimen is designed to fix the first screen of the first group at a minimal distance from the rear surface of the solid carrier of the specimen studied. This distance may vary from 0.01 mm to 10.00 mm. The distance of 0.1 mm is preferable which permits protecting the mirror surface against mechanical damage. The second screen of the first group is placed relative to the rear surface of the specimen solid carrier, at a distance exceeding the distance from the intersection of the boundary of the light flux and the side boundary of the optical cone of the recording system, to the rear surface of the specimen solid carrier. The front surface of the second screen of the first group is equipped with a light absorbing layer. The third screen of the first group is placed after the second screen, and the front surface of the third screen is made as a light-scattering white opaque surface.

Another feature of the invention is that the device has additional first, second, and third screens of the second group placed along the trajectory of the light flux axes at such a distance from the rear surface of the solid carrier that the edge of the screens would not intersect the optical cone of the recording system; the front surfaces of the first, second, and third screens of the second group being made of reflecting, retroreflective and absorbing materials, respectively.

An additional feature of the invention is that the device has attachment points for the first, second, and third screens of the first and second groups, which allow introducing or removing the first and second screens from the trajectory of the optical axis of the recording system.

The attachment point of the first and second screens of the second group makes it possible to replace the screens by removing the screens from the trajectory of the optical axes of the illuminators; or, in addition, has a swivel between the attachment point and the screen holder, and makes it possible to both replace the screens by removing them from the trajectory of the optical axes of the illuminators and turning the mounted screens relative to the trajectory of the optical axes of the illuminators to remove the screens from the light beams upon changing the mode of operation.

Another object of this invention is the method for performing diagnostics tests of a specimen immobilized on a solid carrier or placed in a reaction mixture. In accordance with the method, a diagnostics mode is chosen from a group including measurements of fluorescent and luminescent radiation, light scattering or transmission. One or several screens are in turn introduced to the trajectory of the optical axes of the illuminators and/or to the trajectory of the optical axes of the recording system. The object for analysis is placed into the specimen holder and introduced to the trajectory of the optical axes of the illuminators and the recording system. Shooting conditions are chosen using the preliminary image on the display. The first image of the object is obtained and recorded. The object is removed from the trajectory of the optical axes of the illuminators and the recording system. The second image is obtained and recorded. A differential image of the first and second images is formed. The differential image is pixel-by-pixel multiplied by normalized coefficients, and the program for processing the obtained image is started.

### SUMMARY

The invention relates to a multifunctional device for measuring fluorescence, luminescence and light transmission for diagnostics. The test sample carrier is designed in the form of a biochip, a cell, a pan or a microplate. The device also comprises a first and second groups of screens. Said screens are mounted behind the rear surface of a sample solid carrier and the light sources of the test sample are provided with light absorbing elements for suppressing the light reflected from the front surface of the sample carrier and from the screen surfaces. The holders of the screens make it possible to alternatively mount light reflective and retroreflective screens in such a way that the maximum fluorescent or luminescent signal is provided. A diffusing screen makes it possible to measure the light transmission through the test sample. The light-absorbing screens which are located behind the rear surface of the sample, together with the light absorbing elements, which are located on the light sources from the top surface of the sample, make it possible to increase the signal-to-noise ratio. Said device makes it possible to measure signals on biochip surfaces and in solutions during hybridization or amplification reactions. The device and the method for processing diagnostic data are suitable for mass screening of biological material samples.

### BRIEF DESCRIPTION OF THE FIGURES

FIG. 1. Block diagram of a universal scanner of biochips.

FIG. 2. Block diagram of a device connected to a monitoring system.

FIG. 3. Scheme of formation of a collimated light beam. a) Directional diagram (indicatrix) of LED illumination; b) indicatrix of illumination of LED placed within a black cylinder; c) section of the LED holder.

FIG. 4. Scheme of a device for diagnostics of objects immobilized on the solid surface of a transparent carrier.

FIG. 5. Scheme of a device with combined application of absorbing and reflecting screens for diagnostics of objects immobilized on the solid surface of a transparent carrier.

FIG. 6. Scheme of a device rendering a four-fold enhancement of fluorescent or luminescent signals.

FIG. 7. Scheme of transformation of light incident upon the carrier surface where specimens are layered as clusters with probes. a) Scheme of transformation of incident light with the use of a mirror surface of the screen; b) scheme of transformation of incident light with the use of a retroreflective surface of the screen.

FIG. 8. Scheme of a device rendering scanning of carriers containing objects stained with colorimetric markers.

FIG. 9. Section of a cell for a device working in real-time PCR.

FIG. 10. Block diagram of algorithm for processing the obtained data.

FIG. 11. Images of 13-point clusters layered to the surface of a modified glass chip in two modes of signal recording. a) Measurements without a reflective mirror; b) measurements with a reflective mirror as shown in FIG. 6.

### DETAILED DESCRIPTION

Block Scheme. Upon developing the device it was discovered that the technology tasks of the invention connected with increasing the signal-to-noise ratio and with enlarging the modes of device operation, could be realized by introducing additional screens and providing a possibility to move in, and remove screens from, the trajectories of the optical axes of the device. By this, the signal-to-noise ratio is increased owing to the choice of optical properties of the surface of the given screens, and the choice of modes is widened due to the use of screens with reflecting, retroreflective and light-emitting surfaces.

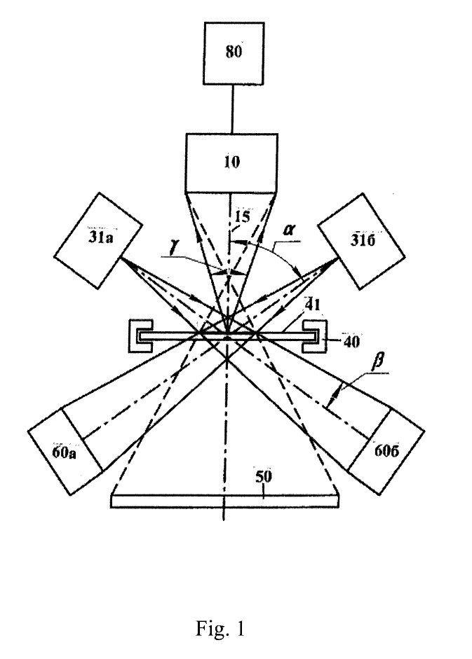

The block scheme of the device is given in FIG. 1. The device consists of an optical receiving system (10) connected to the inlet of the recording and controlling system (80), the first (31a) and second (31b) illuminators forming a light flux with a cone beam angle 2β, optical axes of the illuminators are positioned at angle α to the optical axis (15) of the receiving system (10) with a light gathering angle γ. The device has additional first (50) and second (60a, 60b) groups of screens as well as an attachment point (40) of carriers (41) of the studied specimens.

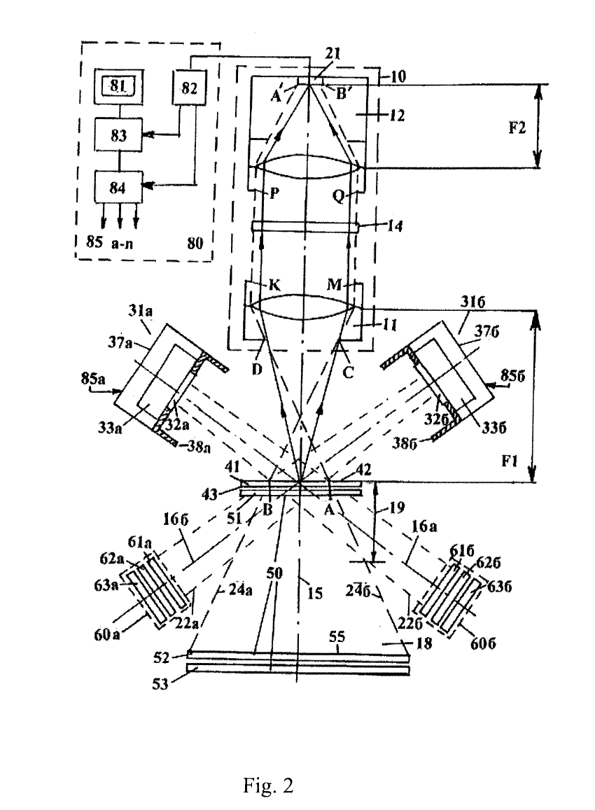

FIG. 2 shows a more detailed block scheme of the device. The device has the following basic elements: an optical system (10) consisting of the first (11) and second (12) parts between which there is the first optical filter (14), a light-sensitive detector (21), a recording and controlling system (80) including a signal-converting circuit (82), a computer (83) for accumulation and processing of the data obtained from the light-sensitive detector (21) as well as for producing monitoring signals for switching off and change-over of the device circuits, for example, the power supply (84). The device contains a display (81), the first (31a) and second (31b) illuminators, an attachment point (40) (not shown in FIG. 2) for the carrier (41) of the studied specimen, the first group of screens (50), and the second group of screens (61a, 61b). The screens of the first group are positioned along the optical axis (15), and the screens of the second group are positioned symmetrically to the trajectory of the optical axis (15) and along the optical trajectories of light fluxes (16a) and (16b) formed by the first (31a) and second (31b) illuminators.

The device operates as follows. The light from the first (31a) and second (31b) illuminators is incident upon the front surface (42) of the carrier (41) of the studied specimen at acute angles lying in the range from (α−β) through (α+β) relative to the optical axis (15). The luminescent light of the specimen is gathered by the optical system (10) and directed to the light-sensitive detector (21). A portion of the light permeates via the transparent carrier (41) of the specimen and comes to the area of the screens included in the first (50) and second groups (61a, 61b). Depending on the chosen operation mode, different types of screens of the first (50) and second (61a, 61b) groups are mounted. A simple replacements or removal of absorbing, reflecting or retroreflective screens from the trajectories of the optical axes is most easily realized, and results in improving the operation of the device.

Centers of the first group of screens (50) are aligned with the optical axis (15) of the device, whereas the surfaces of the screens are positioned perpendicularly to the optical axis of the device. Centers of the second group of screens (61a, 61b) are aligned with the trajectories of the optical axes (16a, 16b) of the illuminators, whereas the surfaces of screens of the second group are positioned perpendicularly to the trajectories of optical axes 16a and 16b.

The second group of screens are mounted relative to the rear surface of the solid carrier of the specimen at a distance (19) exceeding the distance from the rear surface (43) of the specimen carrier (41) to the intersection of the lower boundaries (22a, 22b) of light fluxes and side boundaries (24a, 24b) of the optical cone (18) in the optical system (10). In this case, the light reflected from the screens (60a, 60b) of the second group would not be incident upon the front surface (55) of the second screen of the first group and at the same time the light reflected from the front surface of the screens (60a, 60b) of the second group would not be gathered by the optical system (10).

The carrier (41) of the studied specimen is positioned strictly perpendicularly to the optical axis (15) of the device in such a way that the working area, where the studied specimen is placed, would be located within the field of view AB of the recording optical system (10), and the working surface would be combined with the front focal plane of the first part (11) of the optical system (10). The device for positioning and fixating (40) the carrier (41) of the studied specimen is designed so that it is possible to replace carriers manually or in an automated mode. The field of view AB on the working surface of the carrier (41) is illuminated using two similar sources of excitation fluorescence (31a, 31b) mounted symmetrically to the optical path of the device. The design of the illuminator holders allows manual or automated replacement of the illuminators.

The device is based on dark-field illumination. The optical axis (16a, 16b) of the illuminators makes an acute angle α with the optical axis (15) of the device, at that, the ratio (α−β)>γ/2 is valid. In this case, the excitation fluorescence (including that mirrored from the object) does not get into the optical system (10).

The angle α and the distance from the illuminators (31a, 31b) to the studied specimen can be changed during adjustment of the device for improving the uniformity of illumination. Dashed lines in FIG. 2 show extreme rays AD (24b) and BC (24a) gathered by the recording optics. When absorbing the excitation radiation, fluorochrome molecules bound to the object exhibit fluorescence. The fluorescent light is gathered by the first part (11) of the optical system (10) having the numerical aperture NA=sin(γ/2). The entrance port CD limits the field of view of the optical system. At its outlet, rays are telecentric since the working surface of the object is aligned with the front focal plane of the first part (11) of the optical system. The formation of telecentric rays is required for correct operation of the first interference light filter (14). Then in the case of measuring the fluorescence, the light is transmitted via the band interference filter (14), the spectral characteristics of which are chosen in such a way that on the one hand, it transmits the maximum of the useful signal (the fluorescence of marker), and on the other hand, it provide minimum penetration of stray background illumination to the detector (21).

The latter condition is provided mainly by three circumstances including the following: a) Minimal penetration of the excitation illumination to the recording channel. This depends on the incidence angle of the excitation light beam α, the far field angle β, the quality of the object and mirror surfaces (the absence of stray scattering light), and overall absorption of the excitation illumination within the device. b) Minimal integral of overlapping the transmission spectra of the exciting (32a, 32b) and recording (14) light filters, as well as the ability of the light filter (14) to suppress the excitation illumination. c) Minimal auto fluorescence of the material of the carrier (41) of the object and the light filter (14) in the transmission band of the light filter (14). The design of the device permits replacing light filters (14) manually or in an automated mode. The light transmitted via the light filter (14) is gathered by the second part (12) of the optical system, in the back focal plane of which there is a photosensitive layer of the light-sensitive array (21), for example, a CCD array. It should be noted that the size of the field of view AB depends on the size of the A′B′ image formed on the surface of the array, the ratio AB=A′B′x(F1/F2) where F1 is the focal length of the first part (11) of the optical system, and F2 is the focal length of the second part (12) of the optical system. Parameters of optical systems are chosen in such a way that the A′B′ image would fill completely the sensor array (21), the inlet PQ of the second part (12) of the optical system would be equal to the outlet KM of the first part, and the numerical apertures would be maximally large.

Light sensitive elements of the sensor array (21) convert the light signal into an electrical one. Then this signal is counted, linearly transformed, digitized and transmitted by an electronic device (82) to the computer (83) on the display of which an image of the working area of the object is formed.

Optical System. The first (11) and second (12) parts of the optical system (10) are high-quality optical systems (objective lenses) with highly intensive light transmission that are to a great extent free of geometric and chromatic aberrations. Chromatic aberrations are to a lesser extent capable of affecting the accuracy of measurements since the optics works in quasi-monochromatic illumination from the light filter (14). Inaccuracy of focusing appearing at changing the wavelengths does not affect the work since the depth resolution of the optical system is rather high (about 0.5-0.7 mm).

Most prominent features of objective lenses can include their resolution, contrast transfer coefficient, integral and spectral light transmission factors, light scattering factor and light incidence (light gathering) upon the image field.

Most of the known optical systems for the formation of the first optical system (11) make use of short focus lenses with a small working length. This leads to shortening the distance between the first part (11) of the optical system and the carrier surface (41) of the object for analysis. The distance shortening causes problems upon formation of illumination of the working area. In RU patent 2182328 [24] in one embodiment of the device, the holders of illuminators are mounted directly on the objective lens. This does not allow inserting accessory elements, for example, constructions of cell holders or cell heaters for recording the processes in real time mode, between the first part of the optical system and the surface of the specimen carrier.

In the described device, the first part (11) of the optical system (10) represents a long focus objective lens which permits widening the dimensions of the working area. The dimensions of the working area of the specimen can be varied widely (for example, from 10 through 90 mm). It is expedient to use an industrially manufactured projection or photo lens as the first part (11) of the optical system. Important characteristics are the focus distance, the working distance, the linear field of view, the numerical aperture, the inlet and outlet hatches. For example, the focus distance of photo and projection lenses can vary from 50 to 110 mm, the aperture can vary from 0.17 to 0.26, the field of view from 36×24 mm to 90×60 mm, and the working distance from 45 to 95 mm.

The working distance (the distance from the first lens to the focal plane) of the objective lens 11 should be rather long, not to prevent the transmission of light from the illuminators. Resolution of the first optical system should be no less than 20 lines per 1 mm.

As the second optical system, it is expedient to choose an objective lens designed specially for working with light-sensitive sensor arrays, for example, a TV-lens or a digital camera lens. It is adequate to use a lens with a fixed focus distance (a monofocal lens) and manual aperture setting. It is expedient to use TV-lenses with a focus distance from 25 to 12 mm designed for a sensor array of ⅔″ or ½″. It is necessary to choose high-resolution TV-lenses (megapixel ones) designed for machine vision (with minimum geometrical aberrations).

The objective lens used should be designed for operation with a sensor array of a definite size. However, it can be also used with sensor arrays of a smaller size. For example, a lens labeled as ⅔″ can also work with sensor arrays of ½″, ⅓″, etc. At present, a great variety of monochromatic CCD and CMOS arrays are manufactured with standard sizes from ⅙″ to ⅛″ and so on. The most frequently used are the sizes ⅓″ (the diagonal of 6 mm), ½″ (the diagonal of 8 mm) and ⅔″ (the diagonal of 11 mm). Sensors from 1″ and more are expensive, while sensors from ¼″ and less have a narrow dynamic range and a large noise level.

The linear expansion produced by the system is G=F1/F2. Therefore the focus distance of a TV-lens is chosen based on the dimensions of the working area A′B′ of the object for analysis, the focus distance of the first objective lens and the sensor size. It is important that the inlet hatch PQ of the second objective lens would be approximately equal to the outlet hatch KM of the first objective lens as well as to the light-transmitting diameter of the interference light filter 14.

Interference light filters (32a, 32b) mounted on the illuminators have a transmission bandwidth from 40 to 60 nm. The light filter (14) has a transmission bandwidth from 30 to 50 nm. It is very important that the integral of overlapping of transmission spectra of light filters (32a, 32b) and (14) would be the minimal, because this is decisive for the signal-to-noise ratio of the device. The filters should have guaranteed attenuation of the light outside of the transmission band of 10⁶ and actually 10⁸ (according to the manufacturer). An empirically supported criterion for choosing a pair of filters (14) and (32) is the absence of visible luminescence of light-emitting diodes (LED) appreciated visually in the dark upon superposing the filter (14) on the filter (32) at the illuminator, switched to the maximal power.

The used engineering solutions permit working with CCD arrays without cooling.

Illuminator. To realize the possibility of scanning large surfaces in simple optical systems, it is necessary to achieve maximal uniformity of the object illumination. This is not a trivial task. The use of laser sources is restricted because of sharp non-uniformity of emission across a laser beam, as well as of lamps forming a wide spectrum of the light flux, since it is required that the construction should include elements increasing its dimensions and cost. At the present time, it is preferable to use illuminators based on LED arrays which have low cost and small dimensions.

It is known that LEDs are used for designing fluorescent readers or microscopes for biochip scanning [24, 34]. With the help of LEDs it is possible to provide illumination of the specimen and excitation of its fluorescence for different systems of fluorescence recording. Due to their small size, LEDs can be located close to the objective lens or in its body, or it is possible to use fiber optics for transmission of excitation light from remote light sources, to generate illumination incident at an angle upon the front or rear surface of the biochip, or to form a beam directed perpendicular to the rear surface of the transparent solid carrier [35].

In instruments based on interference optics, it is necessary to form a collimated light beam. This problem is solved by introducing additional collimation lenses [36, 37], parabolic reflectors [38], combinations of slotted screens and cylinder lenses [39], as well as combination of LED arrays with arrays consisting of a variety of lenses [40]. Disadvantages of such solutions may include an additional increase of the cost of illuminators and a larger number of structural elements.

While developing the instrument it was found that a shift of an individual LED from the outer surface of the LED holder into the cylindrical aperture, within which an LED of a cylindrical shape is located having a spherical or parabolic lens at its butt end, makes it possible, when absorbing material is layered on the aperture walls, to form a collimated light beam without employing additional lenses or complex engineering solutions. Thus, part of the parasitic background (which could appear upon irradiation of the object surface with a source scattering light over the ambient surfaces of the illuminator and the specimen holder) is removed.

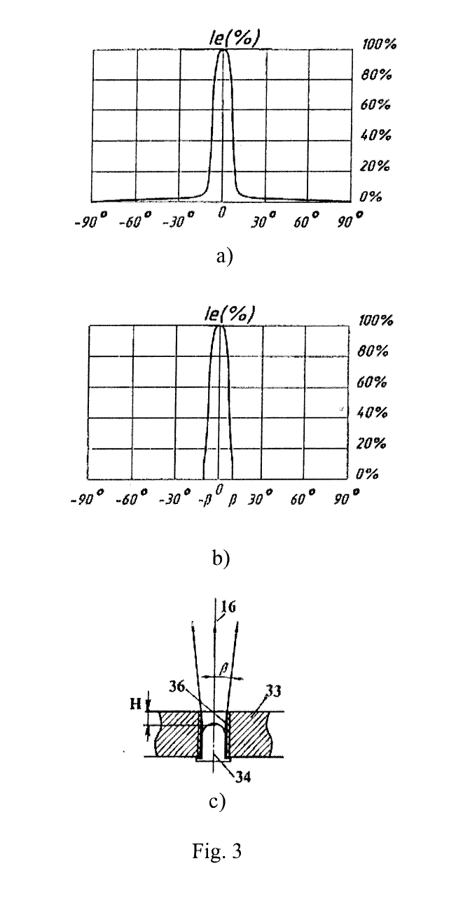

FIG. 3 shows a scheme of formation of a collimated beam employing a black cylinder mounted within the body of the illuminator. FIG. 3a represents the radiation pattern (indicatrix) of emission of an LED (34). FIG. 3b shows the indicatrix of emission of an LED placed in a black cylinder (36) of the LED holder (33) as given in FIG. 3c. Distance H between the front surface of the LED holder (33) and the LED butt end varies from 1 to 5 mm, which permits the formation of a light beam satisfying the condition of a permissible divergence of the beam at which angle β does not exceed the maximum permissible angle indicated in the technical specifications for exploitation of the light filter used (32) (as a rule, the divergence angle β lies in the range from 4 to 7.5 degrees). The length of the aperture (the thickness of the holder) is chosen in such a way that only beams satisfying the condition of permissible beam divergence would be transmitted. That is, angle β should not exceed the maximum permissible angle indicated in the technical specification for exploitation of the light filter used (32) (as a rule, 5 degrees). It should be noted that in the given device, no more than 10% of the emitted light is absorbed.

Each illuminator (31a, 31b) forming a light flux with a definite spectral band of excitation light consists of three basic elements: a light-proof casing (37), an interference light filter (32) and a holder (33) containing an ordered LED array.

The light-proof casing (37) restricts the light beam in such a way that an area slightly exceeding the field of view AB would be illuminated. The casing (37) is coated with a black muted light-absorbing layer (38) from the outside and on the inside.

The holder (33) is a metal plate, the thickness of which is chosen in a special manner. The plate has an ordered set of round through-holes with a diameter corresponding to the LED diameter (preferably 5.0 mm or 3.0 mm). The axes of the holes are perpendicular to the front surface of the holder. The whole surface of the holder (33) and apertures (36) is coated with a black muted light-absorbing layer.

An ordered set of apertures has properties of rotational symmetry of the 6-th or 4-th orders (hexagonal or orthogonal packing). Therefore, maximal density of LED assembling, and (or) illumination uniformity, is achieved. Round LEDs of a standard size (the diameter 5.0 mm or 3.0 mm) are mounted in the apertures. The metal holder (33) is a heat-eliminating element. Upon construction of the illuminator, round apertures permit turning LEDs relative to their long axis at an arbitrary angle and locating their array so as to provide maximal uniformity of illumination of the working area of the object.

The shape and size of an LED array formed in this way are chosen so that its projection to the object plane would approximately correspond to the shape of the working area of the object for analysis. An LED array can form a circle, an oval, a rectangle, a square, a polygon, and a triangle.

At the same time, the shape of the array is chosen with account for the size and shape of serially manufactured interference light filters. A light filter should completely embrace all the apertures in the array without restriction of the light. Preference should be given to light filters of a round shape with the outer diameter of 25 mm, 30 mm, or 50 mm since these are serially produced, and as a result, less expensive.

The light filter (32) is tightly adjoined to the front surface of the holder (33) being positioned perpendicularly to the long axes of LEDs. For this purpose, the LED holder has a round cavity for an accurate fixation of the light filter; simultaneously, the cavity is an optical gate preventing lateral leakage of the LED light.

An illuminator assembled in this way represents a spread radiation source. The uniformity and intensity of illumination of the measuring area is enhanced due to superposition of light spots from every LED. The employment of two illuminators positioned symmetrically enhances light uniformity and power.

The distance from the illuminators (31a, 31b) to the center of the working area where the object for analysis is placed is chosen in such a way that the light spot formed on the surface of the object by one LED would roughly correspond to the minimal size of the illuminated area.

The incidence angle of beams a may vary from 40 to 60 degrees and depends on the parameters (the working distance, the numerical aperture) of the first part (11) of the optical system (10). The larger the angle α (see FIG. 1), the less the distance from the rear surface of the object carrier to the screens (the more compact the construction), but the less the illumination of the object.

The distance between the illuminators (31a, 31b) and the front surface of the carrier (42) as well as angle α can be changed insignificantly during adjustment of the device. After the alignment, optical features of the illuminators (16a, 16b) may deviate from the object center conjugated with the optical axis (15). A preferable distance between the illuminators (31a, 31b) and the object center is about 70 mm at an angle α=55° for objects placed on biochips of 26×76 mm.

The device design allows for changing the illuminators intended for fluorescence excitation from different fluorochromes. Band interference filters (32a, 32b) separate such a spectral range of light that is required for excitation of a fluorescent marker.

Predominant wavelengths of manufactured LEDs of UV and visible light are as follows: 365+375 nm; 405±5 nm; 475±5 nm; 505 nm; 525 nm; 565 nm; 575 nm; 595 nm; 625±5 nm; 660 nm; white light. The LED emission power is chosen in the range from 10 to 25 mW. LEDs are chosen so that their predominant incidence wavelength would be within the filter transmission bandwidth and would maximally correspond to the maximum spectrum of the fluorochrome excitation. The use of an LED array not only enhances the intensity of the excitation light but also increases essentially the uniformity of illumination of the working area of the object.

LED Power Supply. The power supply (84) permits using four modes of LED power supply or their combinations:
1. Series connection of LEDs and their supply with high-stability current. All diodes operate under the same conditions.
2. Series connection of LEDs and their supply with amplitude-stable pulse current of pulse-width modulation (PWM). The current amplitude is maximally admissible for the given LED according to the technical regulations. This allows regulating light intensity of the illuminator (31) without changing the LED illumination.
3. Parallel connection of LEDs and their supply with stable current. This allows adjusting the illumination intensity of every LED to improve uniformity of the illumination.
4. Parallel connection of LEDs and their supply with amplitude-stable pulse current of pulse-width modulation (PWM). This allows both adjusting illumination of every LED and regulating the illuminator (31) light efficiency.

The power supply (84) has a switch electronic chain for synchronizing LED power supply and for signal monitoring the duration of operation of the electron gate of the light-sensitive detector (21). Switching on synchronization allows supplying the LED (illuminate the object) only for a short time of shot exposure (not exceeding 10 s). During this time, the mean supply current in the LED can be enhanced several times (up to 4 times), which in turn results in nearly the same increase of the intensity of the object illumination. Switching off synchronization makes the illuminator operate in a continuous mode, for example, when continuous shot-by-shot input of images to a computer is performed.

Screen Design. In this embodiment, a possibility of changing modes for diagnostics (included in the group consisting of modes for measuring the fluorescence, luminescence, scattering and light transmission) is realized owing to the installation or changing of the screens located along the trajectory of the optical system axis, and/or along the trajectory of light beams formed by light sources. The installed screens permit: a) amplifying the signal-to-noise ratio due to absorption of parasitic light fluxes; b) raising the useful signal level by two-fold transmission of light fluxes via the studied specimen upon reflection and light-return of light fluxes; c) forming various combinations of screens of the first and second groups, which makes it possible to suppress stray illumination and enhance the signal upon formation of a reflected light flux. Such possibilities are realized by alternating the operation and disabling of the screens of the first and/or second groups in the trajectory of the optical axis (15) and/or in the trajectories of light beams (16a, 16b) generated by light sources (31a, 31b).

To achieve this, in this embodiment the first screen of the first group (51) contains a reflective or retroreflective surface. The second screen (52) of the first group has an absorbing surface. The third screen of the first group is made with a light-scattering surface. Screens of the first group placed along the optical axis (15) have a planar surface that is perpendicular to the optical axis (15).

Screens of the second group have reflective, retroreflective or absorbing surfaces. The first screens of the second group with a reflective surface may be designed as planar plates or as a concave spherical or parabolic surface with a linear focus placed perpendicularly to the optical axes (16a, 16b) of illuminators and parallel to the side surface of the carrier (41). The second screens (62a, 62b) of the second group are designed with a retroreflective surface and made of plates with planar surfaces. The third screens (63a, 63b) of the second group have an absorbing surface and can have a planar, concave (cylindrical, parabolic) or angular shape.

Holders of the screens of the first (50) and second (61a, 62b) groups are designed to provide a possibility to put into operation or disable the screens in the trajectory of the optical axes by removing or replacing a screen as well as by turning the screens relative to the optical axis.

Holders of the first screen (51) of the first group can be designed as individual units or constructively combined with the holder of the carrier (41) of the studied specimen. In the case when instead of a planar specimen carrier (41), flat cells are employed for performing hybridization or PCR, the holder of the first screen of the first group can be constructively connected to the attachment point of additional elements which function as controllers of the cell temperature during PCR and/or hybridization.

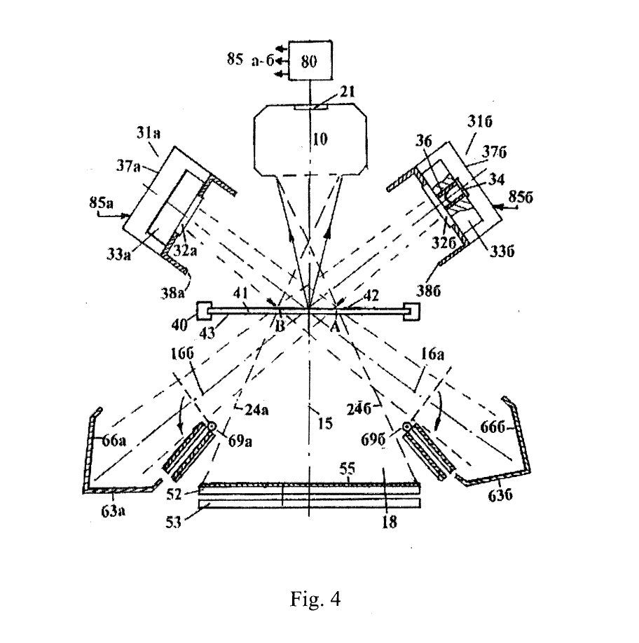

The design of the attachment point of the screens of the second group can have an additional hinge element (69a, 69b) shown in FIG. 4, which provides a possibility of changing the angle of the plane position of the first (61a, 61b) and/or the second screen (621, 62b) relative to the trajectory of the light flux axes (16a, 16b), or the screen holder can be made as a combined construction which can have an attachment point of several screens with a possibility of their individual putting in or removing from the trajectory of the optical axis.

Examples of designing devices (embodiments) for different modes of measurements with different objects for analysis are given below. The examples include, but do not limit other embodiments, that can be designed based on the suggested solutions.

Diagnostics of Objects Immobilized on Solid Surface of Transparent Carrier. FIG. 4 shows an example of an embodiment of the device for diagnostics of objects immobilized on a solid surface of a transparent carrier (41). Such objects may include biochips, tissue sections, and cells. In this embodiment, screens of the first and second groups have an absorbing layer. The device contains an optical system (10) consisting of the first (11) and second (12) parts between which there is the first light filter (14), a light-sensitive detector (21), a recording and controlling system (80) (shown in FIG. 2), the first (31a) and second (31b) illuminators equipped with second light filters (32a, 32b), an attachment point (40) of the carrier (41) of the object for analysis, the second screen (52) included in the first (50) group of screens, two third screens (63a, 63b) included in the second group (60a, 60b) of screens.

Rays are emitted from an illuminator as a divergent beam with angle β. Here, the field of vision is accepted to be a part of the object plane depicted on the light-sensitive detector (21) K′B′. Therefore, in the absence of vignetting, the field of vision has the dimensions G·A′B′, where G=F1/F2 is the value by which the optical system (10) is enhanced. Since light-sensitive detectors have a rectangular shape, the field of vision also has the shape of a rectangle. In FIG. 4, dashed lines (24a, 24b) show extreme rays forming an image on the detector (21). It is obvious that all the rays transmitted inside the cone limited by the dashed line (24a, 24b) will get into the recording optical system (10).

Then the excitation light comes to the working area of the object where it excites fluorescence of a dye(s). A portion of light fluxes passes via the transparent carrier and gets to the space behind the rear surface of the carrier having screens of the first and second groups, which in this mode of recording function as light absorbers.

A significant difference of the engineering solution used in this invention from known systems of parasitic background suppressing [27-30, 41] is the inclusion of light flux absorbers, not just for suppressing the reflected signal from the front surface of the solid carrier on which the object for analysis is placed. In accord with the invention, the device has several levels of parasitic background suppressing. The first level is used for suppressing the reflected light in the construction of holders (33a, 33b) of illuminators where inner surfaces of cylindrical apertures (through which illumination light from individual LEDs is transmitted) are coated with the first absorbent layer.

A light absorbent can be made as an absorbing coating [42] or an absorbing paint because of chemical blackening of the inner surface of an LED holder after making apertures in it. In the latter case, a holder of diodes is made of duraluminium. Apertures are drilled in it for mounting light-emitting diodes, and then it is blackened using known technologies.

The second level of suppressing is provided by absorbing elements (38a, 38b) which serve for suppressing the light illumination reflected from the carrier surface (41) and screen holders of the second group (60a, 60b). The absorbing elements are positioned at the butt ends of the casings (37a, 37b) in which LED holders are fixed. The absorbing elements (38a, 38b) can be of a rectangular or square shape. The surface of the elements (38a, 38b) can be of a planar, concave cylindrical or parabolic shape. It is preferable that the dimensions of the suppressing screen would exceed or be equal to those of the light beam reflected from the surface (42) of the specimen carrier. As an absorbent it is possible to use the butt end of the casing surface (371, 37b) which is covered with an absorbing coating or dye, or the casing surface may be chemically modified for light absorption.

The third level of suppressing of light fluxes that may impair the signal-to-noise ratio is located behind the rear surface of the transparent carrier (41).

The major portion of the excitation light beam that has been transmitted via the solid carrier (41) is suppressed by absorbents (66a, 66b) located on the front surfaces of third screens (63a, 63b) from the second group (60a, 60b) of screens. The screens (63a, 63b) can have the shape of a plate, an angle, a parabola, or a concave cylinder. To ensure the light flux coming to the screens (63a, 63b), the first (61a, 61b) and second (62a, 62b) screens are removed from the trajectories (16a, 16b) of optical paths of the illuminators. The removal can be done by turning the screens (61, 62) around the hinges (69a, 69b).

So that the background excitation emission not absorbed by the absorbers and scattered over the construction elements does not get into the optical system (10), a planar screen (52) having an absorbing layer (55) is positioned in the optical axis trajectory (15). The screen (52) is included in the first group of screens (50). The central part of the screen (52) is aligned with the optical system (10) axis (15).

Generally, the absorbing layer may be prepared using an absorbing material from a group consisting of chemical films, a composition including a carrier and a dispersed pigment, or polymer or textile materials having a sticking layer.

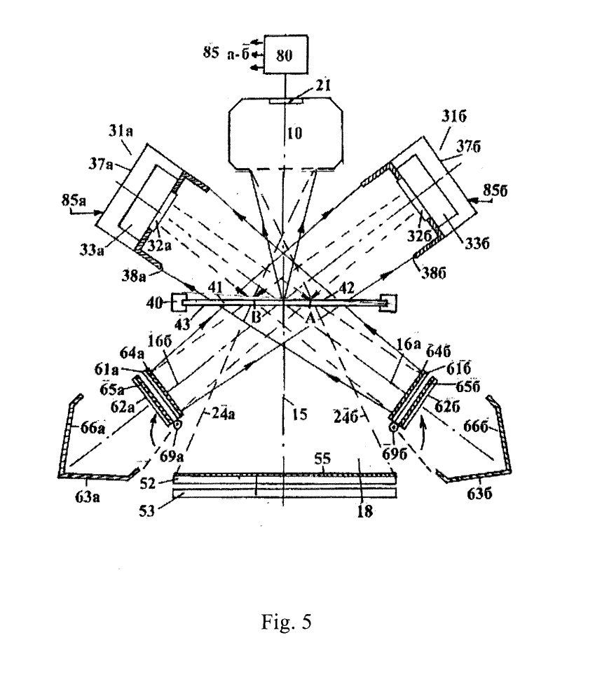

FIG. 5 shows an embodiment of the device with a combined use of absorbing and reflective screens for diagnostics of objects immobilized on the solid surface of the transparent carrier (41). Such objects may include biochips, tissue sections and cells.

Methods are known for increasing the efficiency of taking off data from biochips by using mirror substrates either on the front surface of the biochip [43] or on its rear surface [44]; or a chip is formed as a multilayer structure with inner lenses and a lower reflective surface [45]. A microscope is known [32] in which repeated transmission of a light beam via a transparent specimen for analysis is formed using two objective lenses with identical optical characteristics and a mirror. The mirror is located from the reverse side of the object for analysis behind the second objective lens and reflects the light flux transmitted via the object for analysis.

However this embodiment refers to a certain type of an optical microscope, and it is not proposed to employ it in scanners with a wide working field or in scanners for multi-mode operation. In addition, this embodiment of the device excludes the use of absorbents of scattered light.

This embodiment of the device is based on a combined employment of absorbing and reflective screens. The difference from the above analyzed system shown in FIG. 4 is that incident light beams having been transmitted via the transparent carrier (41) are reflected from the mirror surface of the screens (61a, 61b) which in this embodiment are located perpendicularly to the incident light flux. The front surface of the screens has a reflecting substrate (64a, 64b). The screens (61a, 61b) are moved to the trajectory of the optical path of illuminators (16a, 16b) either with turning mechanisms using hinges (69a, 69b), or the screens are fixed in stationary holders (not shown in FIG. 5).

Being reflected from the mirror surface of the screens, light beams are transmitted once again via the transparent carrier (41) of the object for analysis and then are absorbed on the absorbent surfaces (38a, 38b) placed on the casing of illuminators (31a, 31b). As a consequence, the illumination of the working area of the object is enhanced two-fold. Proportional to the illumination, the fluorescence is enhanced two-fold as well. Uniformity of the illumination is improved essentially. The surface of mirrors does not get into the cone space (18) of the optical system (10). Thus, no artifacts interfere with the imaging. Instead of planar surfaces, it is possible to employ collection mirrors for a better concentration of the reflected beam on the object as it was discussed in the section devoted to the screen design.

The reflective surface (64a, 64b) can be made in the form of a mirror layered onto a glass screen or a mirror spray-coated on a polymer carrier, or can be made as a film with a spray-coated reflective surface having a sticking layer.

The light reflected from the rear surface of the carrier (43) and other structure elements is suppressed on the screen (52), whose surface has an absorbing substrate (55) which contributes to increasing the signal-to-noise ratio.

Another embodiment of the device that can be assembled according to the design shown in FIG. 5 refers to a combined use of absorbing and retroreflective screens for diagnostics of objects immobilized on a solid surface of the transparent carrier (41). In this embodiment, the screens (61a, 61b) are removed from the trajectories (16a, 16b) of light fluxes paths, and the front surface of second screens (62a, 62b) included in the group of second (60) screens located behind the rear surface (43) of the object carrier (41) becomes open.

The front surface of second screens (62a, 62b) is equipped with a retroreflective substrate. The setting of retroreflective substrates allows for returning the light flux incident inside prisms, glass balls or other retroreflective structures. Due to complete internal reflection, the light beam path is refracted within retroreflective elements, after which the flux comes back and gets to the reverse side (43) of the object carrier (41). As a result, the illumination of the working area of the object is enhanced two-fold. Proportional to the illumination, the fluorescence is enhanced two-fold as well.

The enhancement of the intensity of the fluorescent signal exceeds the enhancement of the background, because a remarkable portion of the scattered light definitive for the level of the background signal reduces under the action of the absorbent (55) applied to the screen (52) and the absorbents (38a, 38b) applied to the illuminator casing, which in the end, increases the signal-to-noise ratio.

As a retroreflective layer, it is preferable to use retroreflective materials made in the form of panels, sheets or films with a sticking layer.

The use of retroreflective elements is known for preparing retroreflective panels [46] and retroreflective elements having the shape of a sheet [47]. Flexible retroreflective materials are known which contain a retroreflective structure with a plain front surface and a variety of basic and complimentary retroreflective elements [48] located on its rear surface.

It is most preferable to employ the coatings engineered by firm 3M as retro-reflective materials. The firm offers a wide range of multilayer retroreflective coatings made with the use of spheres [49] and microstructured surfaces [50-52].

The most perspective are retroreflective films of a diamond type. For example, the film of Series 3990 VIP produced by the firm 3M [53] is a material based on microprisms which render a higher retroreflective capacity. Films with a coated retroreflective layer contain a self-adhesive composition and are pasted at room temperature. The most durable warranty period is achieved upon pasting such a film on a preliminarily prepared aluminum surface of the screen.

The retroreflective surface of materials based on the use of cubic angular prisms is made by casting or moulding prismatic elements at the lower surface of a superfine substrate. Dependent on the material type, from 7300 to more than 15500 prisms (20) are allocated per square centimeter.

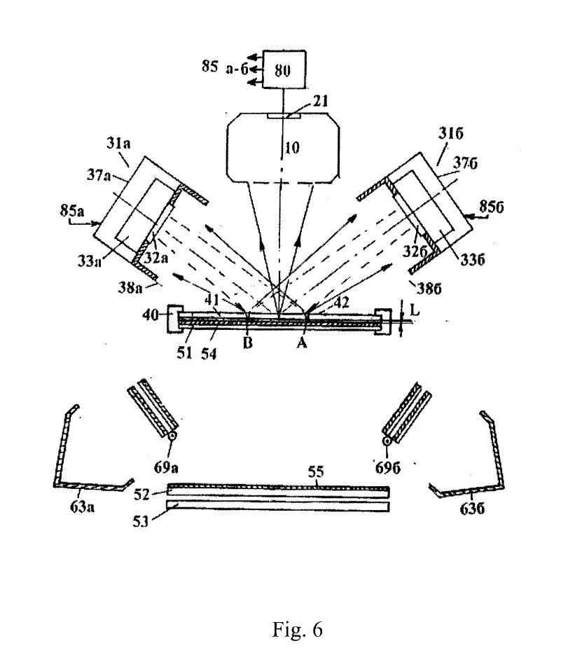

FIG. 6 shows an embodiment of the device that provides a four-fold enhancement of the fluorescent or luminescent signal. In accord with the diagram given in FIG. 6, optical channels contain the first screen (51) included in the first group of screens (50) which overlaps both the trajectory (15) of the optical system (10) and the trajectories (16a, 16b) of optical fluxes from illuminators (31a, 31b). In this embodiment, the screen (51) can contain either a mirror coating or a retroreflective coating. The screen holder (51) can be made as an individual unit or can be constructively combined with the carrier (41) holder of the object for analysis. In this regard, the distance between the rear surface of the carrier (41) and the front surface of the reflective or retroreflective layer are chosen to be the minimum possible, for example, 0.1 mm. The field of vision of the optical system (10) is completely embraced by the mirror or the surface of the retroreflective element. Excitation light is twice transmitted via the object for analysis: in the forward and backward (due to reflectance or light return) directions. The illumination enhances almost two-fold. Furthermore, the fluorescence light emitted towards the mirror is reflected from it and also comes into the recording optical system, which additionally almost doubles the light collection. As a consequence, the general enhancement of the fluorescent light flux increases almost two times. The difference from the known embodiments, for example, from the use of biochips or cells with a mirror surface, is that the signal-to-noise ratio is improved because of the absorption of the scattered light reflected from the upper surface of biochips or cells by absorbing elements (38a, 38b) located on the holders of light sources (31a, 31b).

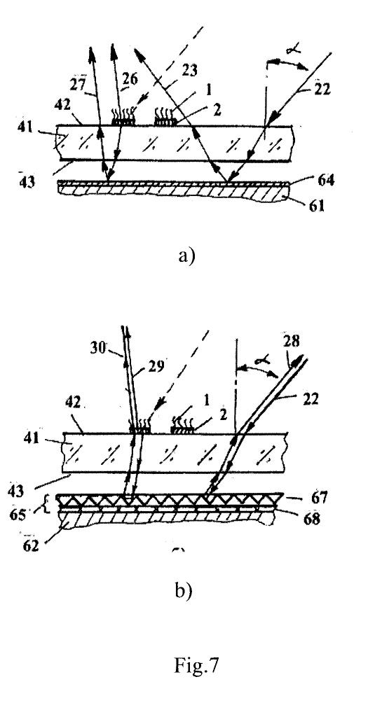

FIGS. 7a and 7b show diagrams of conversion of light (22) incident upon the upper surface (42) of the carrier (41) on which specimens are located as clusters (2) with probes (1), to which molecules with fluorescent markers are hybridized. The diagram of converting the incident light with the use of a mirror surface of the screen is given in FIG. 7a, and the embodiment when the surface of the screen has a retroreflective surface is demonstrated in FIG. 7b.

In the case of using a mirror screen, the light flux (22) having been reflected from the mirror surface, forms a reflected flux (23) that causes additional fluorescence of the specimen. In its turn, the fluorescence signal permeates via the transparent carrier (41) and, having been reflected from the mirror layer (64) on the screen (61), comes back through the back (43) surface of the transparent carrier as the second fluorescence signal (27) in addition to the first signal (26). Thus, the general signal coming to the optical system (10) can be theoretically four times greater than in standard systems of fluorescence scanners.

FIG. 7b shows an embodiment with a retroreflective surface (67) pasted on the screen (62) using a sticking layer (68).

The incident flux (22) having been reflected from the retroreflective surface (67) by the reflected flux (28) induces fluorescence of the specimen again. In turn, the fluorescence signal permeates via the transparent carrier (41) and, having been reflected from the retroreflective layer (67), comes back via the transparent carrier as the second signal (30). Thus, the general signal coming to the optical system (10) can be theoretically four times greater than in standard systems of fluorescence scanners.

However, taking into account the light absorption and scattering on the carrier of the specimen and with consideration of the efficiency of operation of the retroreflective surface that drops with an increase of the incident light (22) inclination angle α, this engineering solution permits the light flux coming back via the rear surface (43) of the carrier (41) as well as enhancing additional illumination of the object not four times, as in the case with a mirror, but from two to three times depending on the type of retroreflective material and the inclination angle α. However, the retroreflective coating is much cheaper than the mirror one.

The mirror or the retroreflective surface gets into the field of vision of the optical system (10). Therefore higher demands are made for the quality of their surfaces. Specifically, the surface should not diffuse the light. Otherwise, the background will be enhanced due to the penetration of excitation illumination into the recording channel. And although the background is eliminated during processing of the image, the dynamic range of the signal becomes narrower. Dust elements absorbed on the mirror surface or on the retroreflective surface are able to diffuse light and become apparent on the primary image (see Procedure for Image Processing). However, they are apparent in the same way in a “blank shot” without an objective lens and are not found in a differential shot.

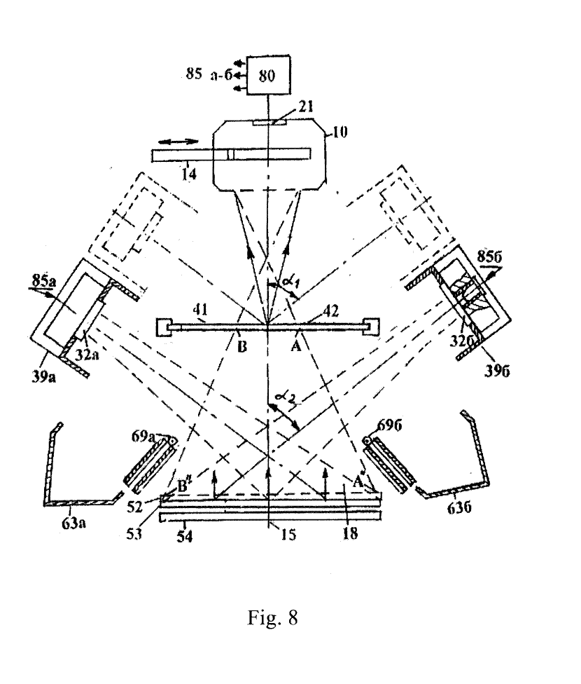

FIG. 8 demonstrates an embodiment which makes it possible to scan carriers with objects stained with colorimetric markers. Configuration is used for transparent objects containing non-fluorescence dyes (for example, biochips with clusters developed during a peroxidase color reaction). In this case, light absorption of a definite wavelength or white light is recorded.

In this embodiment, the light-absorbing screen (52) is removed from the trajectory of the optical axis (15) and a white opaque diffusing screen (53) is introduced into the trajectory. The diffusing screen (53), instead of the light-absorbing screen (52), can be placed in front of the screen (52), or preliminarily placed behind the screen (52). In the latter case, during removal of the screen (52) the front surface of the light-diffusing screen (53) is moved to the trajectory (15) of the optical system (10).

The device has additional third (39a) and fourth (39b) illuminators or a self-luminous (illuminating) screen (54) which provides illumination of the diffusing front surface of the third screen (53) from the front side or from the butt side of the screen (53), or from the back side of the screen (53) as shown in FIG. 8.

Illuminators (39a, 39b) are located so that illumination is incident at angle α₂ directly upon the front surface of the diffusing screen (53), however the standard of dark-field illumination is preserved.

The screen is illuminated uniformly by illuminators (39a, 39b) and diffuses illumination in all directions, including the direction towards the object on the rear surface (43) of the specimen carrier (41). Interference light filters (32) can be present or absent since the level of LED light monochromacity (a band of about 100 nm) proves to be sufficient for some purposes.

In a recoding optical system, the light filter (14) is removed from the trajectory (15) of the beam path. The dominant wavelength of light emitted by illuminators should correspond to the maximum absorption spectrum of the dye. For example, it should be a blue light (the LED dominant wavelength is from 470 to 490 nm) for a color peroxidase reaction with dimethylaminobenzidine.

The object for analysis immobilized on the carrier (41) surface (42) is in the focal plane of the optical system (10). Its image is sharp. The image of the screen (53) surface appears to be greatly diffused (not sharp) and so non-uniformities of illumination are additionally smoothed. When the carrier (41) with the object for analysis is absent in the optical system trajectory, for example, at the stage of measuring the background, the picture frame has a uniform bright background. When the carrier (41) with the object for analysis is moved to the optical system trajectory (10), darker clusters (spots) with dyes are clearly seen against the white background, because during transmission of light via the object, a portion of it is absorbed.

To avoid overloading the light-sensitive detector (21) during measurements, the power of illuminators (39a, 39b) is drastically decreased.

Peculiarities of Objects for Analysis. It is known that biochips are prepared mostly on solid substrates made of glass, polymers, metals, mica and their compositions.

The most popular are biochips on a solid substrate made of microscope object-plates, the dimensions of which are 26×76 mm [54]. The plate surfaces are either modified or probes are immobilized on a non-modified surface. Modification of surfaces with silanes affects only slightly the transparency of slides, therefore glass slides can be used both for the formation of biochips using colorimetric markers [55] and florescent markers [56].

To increase the sensitivity of analysis, it is important that excitation illumination would not induce fluorescence of the material, from which the object for analysis is made. Specifically, when working with glass slides it is expedient to use excitation illumination with the maximum from 625 to 635 nm.

As a carrier of the object for analysis, at least one thin (of 1 mm) standard spectrophotometric cell can be used. In this case, fluorescence of the object solution in the cell or light absorption with the dye at a definite wavelength (the embodiment shown in FIG. 8) is measured. When absorption is measured, a comparison cell can be used. A carrier for the object can be a flow-through cell. The device can operate as PCR in real-time mode (Real Time PCR). In this case, a special PCR cell will be the object carrier. The proposed device can be arbitrarily positioned in the room space, in particular its optical axis (15) can be either in a horizontal or vertical plane. This permit employing both closed and opened cells or microplates.

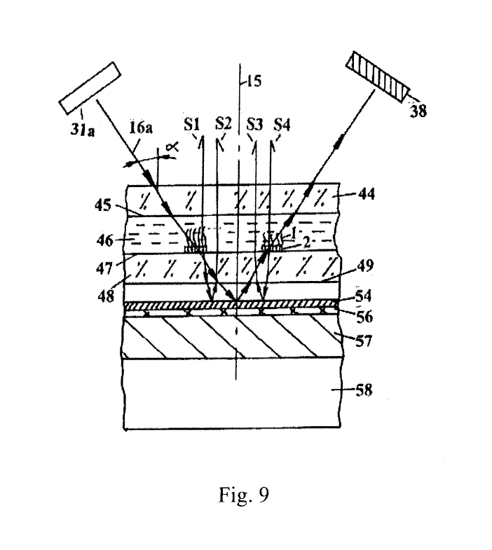

An example of one embodiment of the device, which includes but does not limit other embodiments, is given in FIG. 9.

In accordance with the invention, illumination is directed at angle α to the surface of the cell in which amplification is performed. The light emitted from two illuminators (31a, 31b) is refracted on the front surface (44) of the cell. A portion of the light is reflected from the front surface of the first wall of the cell and comes back to the front surface of the opposite illuminator coated with an absorbing material (38) to eliminate parasitic background. The other portion of the light permeates via the first wall of the cell made of a transparent material and then, having been refracted via the interface between the first inner side (45) of the first wall of the cell and the solution (46), in which amplification is performed, penetrates inside the solution causing fluorescence of markers hybridized with the probes (1), enclosed in biomolecule clusters (2). Having come through the solution, the light is refracted on the second interface between the inner part (47) of the second wall of the cell and the amplification solution. A portion of the refracted light returns to the solution (46). The other portion penetrates via the transparent material (48) of the second wall of the cell and is refracted at the interface between the air space and the rear surface (49) of the second wall of the cell. Having passed via the gap between the rear surface of the cell and the front surface of the first screen of the first group, the light is reflected from the mirror surface (54) of the screen, which is glued with a sticking layer (56) to the cell holder (57), and comes back via the air-gap and the second wall of the cell into the amplification solution. Thus repeated transmission of the light beam via the solution is realized, which enhances the fluorescent signal.

The cell holder (57) has a complementary heater (58) or a Peltier element to control temperature during hybridization or amplification.

The above embodiments of the device do not limit other embodiments of locating other objects immobilized, for example, on a microboard. To simplify the user's work, the device can be fixed in a vertical position, in a horizontal position or can operate when its optical system is placed in the lower part of the device, so that to illuminate the rear surface of microboards.

Description of Image Processing. Depending on the tasks of diagnostics for measuring fluorescence or luminescence, it is advisable to use the first screen of the first group that is placed in the object holder, or the second screen of the first group combined with the first or second screens of the second group, or the second screen of the first group combined with the third screens of the second group. To measure the transmission or scattering, it is advisable to use the third screen of the first group combined with the third screens of the second group.

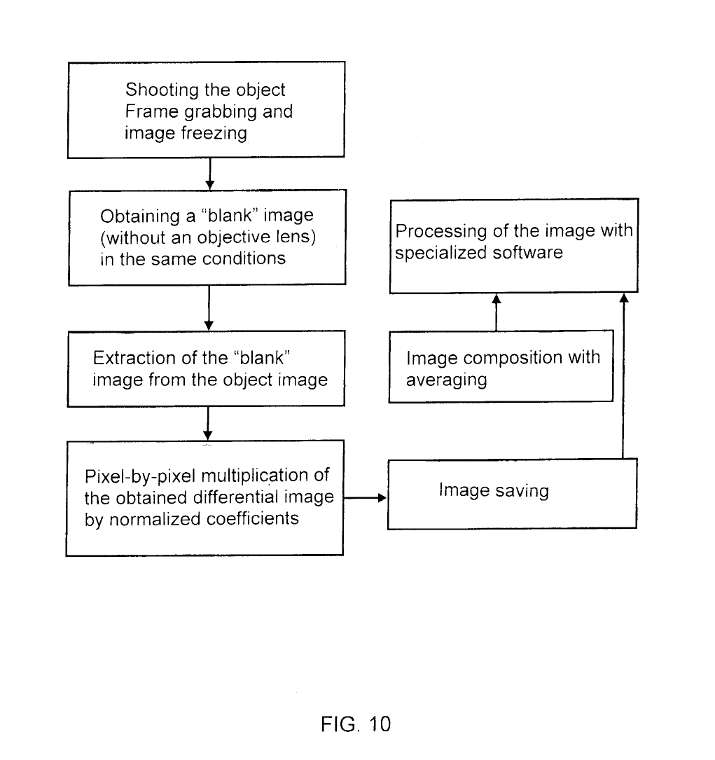

An algorithm of the method for data processing is given in FIG. 10. The final image of the object is formed as follows. The object for analysis is placed in the device. Based on a preliminary image on the computer display (81) and using the device (82), shooting conditions are chosen (exposition duration, signal enhancement) so that saturation of pixel brightness of the light-sensitive detector (12) would not take place.

The image is captured by the computer. The shot with the image is saved. This shot contains both a useful signal from the object and a noise signal superimposed on it.

Then the object is removed from the device and a “blank shot” is made under the same conditions. The blank shot contains information only about the noise signal since there is no useful signal. The blank shot records weak background light, luminescence from dust elements on the mirror and other units of the optical system, heat noises and “hot pixels” of the light-sensitive detector, a steady-state component (“black level” shift) in the signal and other interferences independent of the object.

Furthermore, the “blank shot” is extracted from the basic shot and the result is saved. This procedure minimizes errors in measuring. It is correct since signal conversion in the device (82) is linear.

The obtained differential shot is pixel-by-pixel multiplied by corresponding normalized coefficients to align the image over the field of view AB, to take into account (compensate for) non-uniformity of illumination of the field of view with excitation light and non-uniformity of fluorescence light collection with the recording system (10).

The shot finally formed as described above can be saved to the computer. Several shots from the same object can be summed up with averaging in order to decrease random noises, and after that, processed with corresponding programs using specified algorithms.

Note that the procedure of image processing in a photometric embodiment is the same as that in a fluorescent embodiment. The image with the object (with dark spots) is extracted from the first image without the object (bright background). As a consequence, a negative (light spots against a dark background) image of the object appears, which is then leveled with multiplication by a normalized coefficient.

Obtaining Normalized Coefficients. To estimate normalized coefficients, it is necessary to use a reference sample. It is assumed that a reference sample has an ideally uniform distribution of the fluorescent light emission density over its surface. In our case, a reference sample should represent a fine (no more than 0.5 mm) transparent layer uniformly fluorescing over the area. This layer should be fixed on a transparent non-fluorescing carrier analogous to the biochip carrier. For example, it can be a fine (<0.1 mm) uniformly-thick (no less than 1%) transparent fluorescing film fixed to the surface of a non-fluorescing plate made of CONFIDENTIAL ATTORNEY WORK PRODUCT ATTORNEY CLIENT PRIVILEGE plastic, optical glass or quartz. Or it can be a thin parallel-sided plate made of stained fluorescing optical glass. Or it can be a layer of liquid containing fluorescing molecules and placed between two strictly parallel transparent non-fluorescing plates. The distance between the plated is about 0.1-0.2 mm. Or it can be a molecular layer of fluorochrome immobilized on the surface of a transparent non-fluorescing plate with strictly uniform distribution over the surface.

Note that, theoretically in the absence of device errors, the brightness of emission of all pixels of the image of a reference sample should have been the same. However, owing to non-uniform illumination of the sample with the illuminators (31), non-uniformity of light fluorescence collection by the optical system (10), and other reasons (the presence of excitation light background, the existence of errors of light refraction into an analog electrical signal and its analog-to-digital conversion, electron noise), the brightness of pixels of the reference object image is different.

The pixel-by-pixel normalization allows for essentially decreasing (no less than 15 times) the general error of measurements. The procedure comprises the following: One or several (which is more preferable) reference samples are surveyed. When processing reference images, pixel-by-pixel multiplication by normalized coefficients is not performed or they are supposed to be equal to unity. The obtained shots are summed up with the averaged one (i.e. are summed up pixel-by-pixel and divided by the number of shots, if required the image is smoothed according to known mathematical procedures). As a result, an averaged reference shot is obtained characterizing for the most part non-uniformity of the sample illumination by illuminators (31) and non-uniformity of light collection (at the edges of the field of vision) of the recording system (10).

The brightness value for the “reference” pixel for which normalization will be done is chosen. This can be the brightest pixel of the reference shot or the average brightness value of the shot, etc.

For each pixel of the reference shot, the coefficient is calculated which is equal to the result of division of the brightness value of the “reference” pixel by the brightness value of the given pixel. It is evident that upon pixel-by-pixel multiplication (every pixel multiplied by its coefficient) by these normalized (alignment) coefficients, the brightness values of all pixels of the reference shot will be equal to the value of the “reference” pixel. So, the image is aligned by the field of view.

The estimated normalized coefficients are saved as an ordered array (a normalization factor) which is a peculiar “passport” of the device and is saved during the whole time of its operation.

In a photometric embodiment, the reference sample image is not used when calculating normalized coefficients. As an alternative an opaque screen image is used.

For every pair of illuminators, its own array of normalized coefficients is calculated. The coefficients are calculated individually for every device, are saved and used during the whole period of its exploitation. In case the device is readjusted, for example, because of a repair, its normalization factors are recalculated.

Signal-Converting Circuit. The signal-converting circuit (82) comprises a circuit for monitoring the operation of the detector (21), performs analog-to-digital conversion of the brightness signal from every cell of the detector (21), controls shooting conditions (shooting exposition, enhancement, etc.), interacts with the computer (data organization and transmission) by a special interface (e.g. USB bus), synchronizes the operation of illuminators controlling the work of power sources.

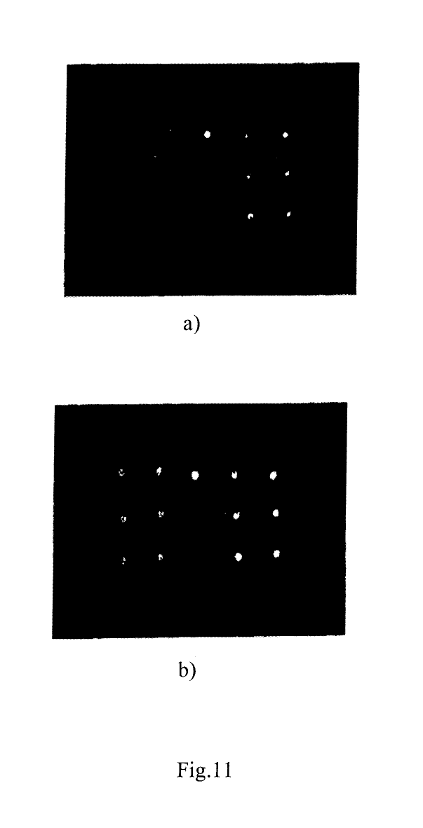

FIG. 11 shows a cluster consisting of 13 dots layered onto the surface of a modified glass chip in two embodiments of signal measuring.

In the first embodiment, a diagram of the device given in FIG. 4 was used. Two light fluxes from light sources (31a) and (31b) illuminate the surface of a glass slide (41) with layered probes and form a dark field at which the emitted flux does not get into the optical system (10). The reflected emission from the slide surface is absorbed on the surface of suppressing elements (38a) and (38b). The light fluxes pass via the transparent slide with the layered specimen and are absorbed by suppressors (66a) and (66b) located perpendicularly to the trajectory of the light fluxes. In addition, the scattered light is suppressed on the surface (55). The use of such an arrangement permits diminishing the background and obtaining an image of the dots with probes at the maximal signal-to-noise ratio given in FIG. 11a.

In the second embodiment of signal measuring, the diagram of the device given in FIG. 6 was used. In this embodiment, the device has a mirror (54) located behind the rear surface of the slide. This engineering solution makes it possible to increase the signal level up to four times. The result of such an enhancement of the signal is shown in FIG. 11b.

### INDUSTRIAL APPLICABILITY

The device is designed for recording fluorescence of fluorochrome(s) immobilized on the surface or in a thin layer of an object as well as for measuring absorption or scattering of colorimetrically stained clusters of biochips.

This device can operate with transparent, semi-transparent, opaque, black and mirror surfaces. The design of the device does not envisage mechanical scanning of the specimen for analysis in the X-Y coordinates.

The device can work as a fluorimeter and a photometer, i.e. it can measure solution fluorescence and absorption. For example, it can be used to determine the concentration of DNA, protein and other substances in solution or to control the quality of the isolated DNA obtained in PCR.

The device permits performing kinetic measurements with specific times depending on the operation speed of the electron mechanism (82) (typically it is 0.1 s). To this end, the mode of continuous input of images (e.g., an image flow) is used with the processing of every image following.

The device can operate at an arbitrary orientation in the room since the object is fixed in a special holder.

A device constructed in accordance with the invention, has a number of significant advantages over known designs. It has a very simple design, without high requirements to the optical components used, aid, which is of special importance, does not make any specific conditions for the inlet of excitation emission of fluorochrome and/or the outlet of fluorescent emission. Moreover, the device permits performing diverse biochemical studies, and preparation of its basic elements is not highly expensive.

### REFERENCES

1. Peukert M. et al. Device for measuring in particular luminescent and/or fluorescent radiation. U.S. Pat. No. 6,949,754 (Sep. 27, 2005).
2. Froot H. Fluorescent microanalytical system and method for detecting and identifying organic materials. U.S. Pat. No. 4,087,685 (May 2, 1978).
3. Perov A. et al. Biochip scanner device. U.S. Pat. No. 6,329,661 (Dec. 11, 2001).
4. Trulson M. et al. Method and device for imaging a sample on a device. US Patent Appl. 20050281708 (Dec. 22, 2005).
5. Giebeler R. et al. Optical system for a scanning fluorometer. U.S. Pat. No. 6,236,456 (May 22, 2001).
6. Modlin D. et al. Multi-mode light detection system. U.S. Pat. No. 6,825,921 (Nov. 30, 2004).
7. Fernandes J. et al. Multi-functional photometer with movable linkage for routing optical fibers. U.S. Pat. No. 5,436,718 (Jul. 25, 1995).
8. Gambini M. et al. Instrument for monitoring polymerase chain reaction of DNA. U.S. Pat. No. 7,008,789 (Mar. 7, 2006).
9. Bickel R. et al. Device for the amplification and detection of nucleic acids. US Patent Appl. 20060078929 (Apr. 13, 2006).
10. Remacle J. et al. Real-time PCR of targets on a micro-array. WO2006053770 (2006 May 26).
11. Perov A. 14. Perov A. et al. Portable biochip scanner device. U.S. Pat. No. 6,407,395 (Jun. 18, 2002).
12. Barsky V. E. Fluorescent microscope. RU Patent 2166201 (2001 Mar. 27).
13. Yokokawa N. et al. Method and device for reading fluorescence. U.S. Pat. No. 6,646,271 (Nov. 11, 2003).
14. Mathies R. et al. Laser excited confocal microscope fluorescence scanner and method. U.S. Pat. No. 5,091,652 (Feb. 25, 1992).
15. Hueton I. et al. High-speed fluorescence scanner. U.S. Pat. No. 5,459,325 (Oct. 17, 1995).
16. Kureshy F. et al. Microarray detectcr and methods. U.S. Pat. No. 7,354,389 (Apr. 8, 2008).
17. Wang J. et al. Fluorescent microarray analyzer. U.S. Pat. No. 7,042,565 (May 9, 2006).
18. Tanaami T. Biochip reader and fluorometric imaging device. US Patent Appl. 20060029523 (Feb. 9, 2006).
19. Wang J. et al. CCD-based biochip reader. U.S. Pat. No. 7,173,701 (Feb. 6, 2007).
20. Katerkamp A. Device and method for quantitative affinity analysis using fluorescence markers. RU Patent 2158916 (2000 Nov. 10).
21. Bevis C. et al. Darkfield inspection system having photodetector array. U.S. Pat. No. 7,061,598 (Jun. 13, 2006).
22. Peterson T. et al. Use of light scattering particles in design, manufacture, and quality control of small volume instruments, devices, ard processes. U.S. Pat. No. 6,714,299 (Mar. 30, 2004).
23. Oldham M. et al. Combination reader. U.S. Pat. No. 6,970,240 (Nov. 29, 2005).
24. Barsky V. E. et al. Fluorescent microscope. RU Patent 2182328 (2002 May 10).
25. Montagu J. et al. Reading of fluorescent arrays. US Patent Appl. 20060127946 (Jun. 15, 2006).
26. Albertson D. G. et al. Wide field image for quantitative analysis of microarrays. US Patent Appl. 20050260741 (Nov. 24, 2005).
27. Chhibber R. et al. Method and device for illuminating a substrate during inspection. US Patent Appl. 20050146719 (Jul. 7, 2005).
28. Xu J. et al. Defect review system and method. US Patent Appl. 20050094136 (May 5, 2005).
29. Engelhardt J. et al. Optical system in the ray path of a confocal fluorescence microscope. U.S. Pat. No. 6,785,302 (Aug. 31, 2004).
30. Ince C. System and method for imaging the reflectance of a substrate. US Patent Appl. 20060241364 (Oct. 26, 2006).
31. Mueller G. et al. Optical system for transmitted-light microscopy with incident illumination. U.S. Pat. No. 4,515,445 (May 7, 1985).
32. Mueller G. et al. Microscope for reflected-light and transmitted-light microscopy. U.S. Pat. No. 7,081,994 (Jul. 25, 2006).
33. Ellis G. et al. Confocal laser scanning transmission microscope. U.S. Pat. No. 5,035,476 (Jul. 30, 1991).
34. Yershov G. et al. Biochip reader with enhanced illumination and bioarray positioning device. U.S. Pat. No. 6,620,623 (Sep. 16, 2003).
35. Douglas-Hamilton D. et al. Motility scanner and method. U.S. Pat. No. 4,896,967 (Jan. 30, 1990).
36. Hart S. Light emitting diode (LED) array for excitation emission matrix (EEM) fluorescence spectroscopy. U.S. Pat. No. 6,985,224 (Jan. 10, 2006).
37. Panagotacos G. et al. Illuminator assembly. U.S. Pat. No. 7,163,318 (Jan. 16, 2007).
38. Stopa J. et al. Led light assembly. US Patent Appl. 20030156416 (Aug. 21, 2003).
39. Tsikos C. et al. Planar LED-based illumination array (PLIA) chips. U.S. Pat. No. 6,959,870 (Nov. 1, 2005).
40. Lee K. LED package, display panel, illumination system and projection system employing the same. US Patent Appl. 20060146297 (Jul. 6, 2006).
41. Rao N. et al. Optical method and device for inspecting large area planar objects. US Patent Appl. 20020088952 (Jul. 11, 2002).
42. Harris D. et al. Composition of coating absorbing UV radiation. RU Patent Application 2003133667 (2005 May 10).
43. Garini Y. Optical detection method for improved sensitivity. U.S. Pat. No. 6,552,794 (Apr. 22, 2003).
44. Microarray High Sensitivity Mirror Substrates. MaTep [symbol] a [symbol] [symbol] pM [symbol] TeleChem International Inc. (http://arrayit.com/Products/Substrates/Mirror/mirror.html).
45. Matsushita T. et al. Component, device, and method for analyzing molecules. U.S. Pat. No. 6,999,166 (Feb. 14, 2006).
46. Veligdan D. Light-redirecting panel. RU Application 2001104428 (2003 Apr. 10).
47. Araki Y. Light-emitting retroreflective sheet and method for its preparation. RU Patent 2204154 (2003 May 10).
48. Molokhina L et al. Flexible retroreflective material. RU Patent 2183336 (2002 Jun. 10).
49. Mori Y. Retroreflective sheet comprising microspheres, the diameter and refractive index of which being specifically related to the refractive indices of layers directly in contact therewith. U.S. Pat. No. 5,962,121 (Oct. 5, 1999).
50. Smith K. et al. Cube corner cavity based retroreflectors with transparent fill material. US Patent Appl. 20010033907 (Oct. 25, 2001).
51. Smith K. et al. Flexible cube-corner retroreflective sheeting. US Patent Appl. 20020126382 (Sep. 12, 2002).
52. Smith K. Retroreflective sheeting having high retroreflectance at low observation angles. US Patent Appl. 20070081245 (Apr. 12, 2007).
53. 3M Diamond Grade VIP Reflective Sleeting Series 3990. Tex [symbol] ecKoe o [symbol] ca [symbol] e [symbol] pM [symbol] Minnesota Mining and Manufacturing (3M) Product Bulletin 3990 November 2003 (http://multimedia.mmm.com/)
54. Zeleny R. et al. Automatic imaging and analysis of microarray biochips. U.S. Pat. No. 6,215,894 (Apr. 10, 2001).
55. Beletsky I. P. et al. Set of primers for detection and/or identification of transgenic DNA sequences in plant material and products containing such material (embodiments), paraprimers (embodiments), method for detection and/or identification with their use (embodiments) and device for realization of the method. RU Patent 2265668 (2005 Dec. 10).
56. Mirzabekov A. D. et al. Method for identification of transgenic DNA sequences in plant material and products based on it, set of oligonucleotides and biochip for realization of this method. RU Patent 2270254 (2006 Feb. 20).

What is claimed is:

1. A device for measuring fluorescence, luminescence, scattering and transmission of light for diagnostics. Said device comprising at least two light illuminators that form illumination of the working field, an optical system, a detector, an attachment point for a specimen, a solid carrier of the specimen for analysis, wherein a first and second group of screens are present, the first group having at least one screen and the second group having at least two screens, where the screens are placed behind the rear surface of the specimen solid carrier, and the illuminators contain absorbents for suppressing the reflected illumination from the front surface of the specimen carrier and the surfaces of the screens, where the screens of the first group are positioned perpendicularly to the optical axis of the recording system and the screens of the second group are positioned perpendicularly to the optical axes of the illuminators.

2. The device of claim 1, wherein the first screen from the first group is made so that it can reflect or retroreflect the light fluxes of the first and second illuminators and is positioned at a minimal distance (from 0.01 through 10.00 mm) from the rear surface of the object solid carrier, where the front surface of the first screen has a reflective or retroreflective layer.

3. The device of claim 3, wherein the attachment point of the holder for the object solid carrier provides a possibility to position the first screen of the first group behind the rear surface of the solid carrier and to remove it from the field of view.

4. The device of claim 1, wherein a second screen of the first group is positioned relative to the rear surface of the object solid carrier at a distance exceeding the distance from the point of intersection of the lower flux boundaries and side boundaries of the optical cone of the recording system, where the front surface of the second screen of the first group has a light-absorbing layer.

5. The device of claim 1, wherein a third screen of the first group is placed behind the second screen of the first group, where the front surface of the third screen is made as a light-scattering surface.

6. The device of claim 1, wherein there is an additional attachment point for the second and third screens of the first groups and it is possible to remove the second screen from the area of the optical cone of the recording system.

7. The device of claim 1, wherein there is at least one additional third light source, where the third light source illuminates the front surface of the third screen, the butt-end surfaces of the third screen, or the rear surface of the third screen.

8. The device of claim 1, wherein there are additional attachment points for the first and second screens of the second group which make it possible to move in and remove the screens from the trajectory of the optical axes of the illuminators, where the attachment point of the first and second screens of the second group is made using a hinge joint between the attachment point and the screen, and it is possible to turn the screens relative to the optical axis of the illuminator.

9. The device of claim 1, wherein the first screens of the second group have a light-reflective layer, and the second screens of the second group have a retroreflective surface.

10. The device of claim 1, wherein there are additional third screens of the second group which are positioned behind the first and second screens of the second group, the front surface of the third screens having an absorbing layer.

11. The device of claim 1, wherein the screen is a planar, angular, cylindrical or parabolic element with a reflective, light-absorbing or retroreflective surface.

12. The device of claim 1, wherein the light from the light sources is incident upon the working surface of the object for analysis at an angle α to the optical axis of the recording system in the range from 40 to 60 degrees.

13. The device of claim 12, wherein the light source has an additional light-absorbing coating layered onto the surface of holders with cylindrical apertures, within which light diodes and light-absorbing elements are fixed, that are positioned on the surface of the illuminator casing, where light-absorbing elements have a planar, concave, cylindrical or parabolic shape and where the light source emits illumination in the range from 300 through 800 nm.

14. The device of claim 1, wherein the solid carrier of the specimen for analysis is made as a biochip, a cell, or a microplate, where the specimen for analysis is a biological sample immobilized on a solid planar substrate, a sample placed within a flow-through cell, a sample placed within a hybridization solution, a sample layered on a flexible substrate pasted to a solid planar substrate, a sample immobilized on a gel substrate, a sample fixed on a chromatographic carrier, and a biological sample chosen from a group comprising DNA, proteins, enzymes, antibodies, antigens, and cells.

15. The method for performing diagnostic tests by illuminating a specimen immobilized on a solid carrier or placed in a reaction solution, wherein:
a) The mode of diagnostics is chosen from a group including measurements of light fluorescence, luminescence, scattering or transmission;
b) One or several screens are in turn introduced into the trajectory of optical axes of illuminators and/or in the trajectory of the optical axis of the recording system;
c) The object for analysis is placed in the object holder and it is introduced into the trajectory of optical axes of illuminators and the recording system;
d) Based on the preliminary image on the display, shooting conditions are chosen and the first image is saved;
e) The object is removed from the trajectories of the optical axes of the illuminators and the recording system;
f) The second image is saved;
g) A differential image of the first and second images is formed;
h) The differential image is multiplied pixel-by-pixel by the normalized coefficients and the processing of the obtained image is started.

16. The method of claim 15, wherein the first screen of the first group placed in the sample holder is used for measuring fluorescence or luminescence.

17. The method of claim 15, wherein the second screen of the first group combined with the first or second screens of the second group is used for measuring fluorescence or luminescence.

18. The method of claim 15, wherein the second screen of the first group combined with the third screens of the second group is used for measuring fluorescence and luminescence.

19. The method of claim 15, wherein the third screen of the first group combined with the third screens of the second group is used for measuring transmission or scattering.

20. The method of claim 15, wherein a transparent layer uniformly fluorescing over the area is used as a reference object for estimating the normalized coefficient, where the fluorescing layer is a film fixed on a plastic, optical glass or quartz carrier.
